(19) United States
(12) Patent Application Publication
(10) Pub. No.: US 2015/0027439 A1
(43) Pub. Date: Jan. 29, 2015

(54) ANESTHETIC VAPORIZER

(71) Applicants: Michael Hutchens, Portland, OR (US); Alkayed R. Nabil, II, West Linn, OR (US); Henry Casson, Portland, OR (US); Katie Schenning, Portland, OR (US)

(72) Inventors: Michael Hutchens, Portland, OR (US); Alkayed R. Nabil, II, West Linn, OR (US); Henry Casson, Portland, OR (US); Katie Schenning, Portland, OR (US)

(73) Assignee: OREGON HEALTH AND SCIENCE UNIVERSITY, Portland, OR (US)

(21) Appl. No.: 14/444,785

(22) Filed: Jul. 28, 2014

Related U.S. Application Data
(60) Provisional application No. 61/859,462, filed on Jul. 29, 2013.

(51) Int. Cl. A61M 16/10; A61M 16/18; A61M 16/00

(52) U.S. Cl. CPC A61M 16/109; A61M 16/0051; A61M 16/18; A61M 16/0003
USPC 128/200.11; 128/203.14

(57) ABSTRACT

Disclosed herein is an anesthetic vaporizer comprising a refrigeration system configured to cool a liquid anesthetic to less than 0° C. and to the anesthetic's temperature of minimal alveolar concentration. Further disclosed are methods of administering a liquid anesthetic comprising cooling the liquid anesthetic to less than 0° C. and to the anesthetic's temperature of minimal alveolar concentration.

### CROSS REFERENCE TO RELATED APPLICATIONS

The present application claims priority to U.S. Provisional Patent Application No. 61/859,462, filed Jul. 29, 2013, entitled “ANESTHETIC VAPORIZER,” the entire disclosure of which is hereby incorporated by reference in its entirety.

### ACKNOWLEDGEMENT OF GOVERNMENT SUPPORT

This invention was made with United States government support under the terms of grant number DK090754-01, awarded by the National Institutes of Health. The United States government has certain rights in this invention.

### FIELD

Generally, the field is devices that deliver anesthetic. More specifically, the field is devices that deliver anesthetic vapor.

### BACKGROUND

Currently available anesthetic vaporizer technology requires frequent calibration and maintenance, with each calibration unique to a particular anesthetic. Such vaporizers are also heavy and slow to change concentration. This makes use of currently available anesthetic vapor technology difficult in non-operating room contexts such as animal research laboratories, intensive care units, other austere environments, and military environments including battlefield environments.

### SUMMARY

Described herein is an anesthetic vaporizer that uses the temperature-vapor pressure relationships of volatile anesthetics to deliver clinically relevant concentrations of anesthetic vapor. One significant drawback of variable bypass vaporizers is the requirement of regular servicing and calibration. This complicates delivery of anesthesia in several settings. In animal research laboratories, Institutional Animal Care and Use Committees generally require that anesthetic vaporizers be serviced annually (Vogler G A, Anesthesia Delivery Systems in Anesthesia and Analgesia in Laboratory Animals, 2^{nd} Ed, Academic Press, Fish R E et al, eds pp 127-169 (2008); incorporated by reference herein.) For this service, the vaporizer generally needs to be shipped to the manufacturer or other authorized service center where the vaporizer is disassembled, inspected, cleaned, and calibrated. A temperature controlled vaporizer would not require this level of servicing.

Delivery of a reliable concentration of anesthetic vapor is challenging in suboptimal situations such as in rural clinics in developing nations. Often, anesthesia machines and vaporizers are donated to these sites, but proper maintenance and calibration of the technology is challenging. Actual delivered concentration of anesthesia may be different than set concentration, and end-tidal anesthetic monitoring may be unavailable (Tobias J D, et al, South Med J 95, 239-247 (2002); incorporated by reference herein). Variable bypass vaporizers are agent-specific, and can only be used when a particular anesthetic agent is available. The use of the temperature-controlled vaporizer described herein would be universal across a wide range of anesthetics and therefore provides a more efficient system for volatile anesthetic delivery.

Disclosed herein is an anesthetic vaporizer that includes a vessel, a refrigeration system, and a controller, e.g., an electronic controlling device or other suitable computing system. The vessel may be any vessel configured to contain a liquid anesthetic. Said vessel can also comprise a fresh gas inlet, a mixed gas outlet, and a temperature probe. The refrigeration system can be any refrigeration system configured to cool the anesthetic in the vessel to less than 0° C. The refrigeration system can further comprise a coupling apparatus configured to receive the vessel, a cooling bath to cool the coupling apparatus, and a power source to power the refrigeration system. In some examples, an input device may be coupled to and/or integrated with the controller and configured to allow a user to input a desired parameter such as a desired anesthetic concentration or temperature into the system. Additionally, in some examples, a display device, e.g., a screen, may be coupled to and/or integrated with the controller to communicate or display data to the user. The controller may include machine-readable instructions, e.g., software, which is configured to perform one or more of the following features either alone or in combination: provide an interface configured to allow the user to set a first concentration of the anesthetic, implement an algorithm that calculates the set temperature to achieve the first concentration, and implement a proportional-integral-derivative controller to maintain the liquid anesthetic at the set temperature.

Also disclosed herein is a method of administering an anesthetic to a subject that involves cooling the anesthetic to less than 0° C. and administering the cooled anesthetic to the subject with an anesthetic vaporizer. In some examples, the anesthetic is cooled to the temperature of minimal alveolar concentration of the anesthetic. This method can further comprise calculating the temperature of minimal alveolar concentration for the liquid anesthetic. Additionally, in some examples, the cooled anesthetic output by the anesthetic vaporizer may be heated to a threshold temperature prior to delivery to the subject.

Using the disclosed system, the amount of anesthetic delivered to a subject is controlled by controlling the temperature of the anesthetic rather than air flow or other parameters, thereby a) forgoing the need for servicing and calibration b) providing use of a universal system for a wide variety of liquid anesthetics, and c) allowing measurement of the specific heat of an unknown and/or mixture of anesthetics in a vessel thereby identifying the contents of the vessel.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a schematic depiction of an example embodiment of an anesthetic vaporizer in accordance with the disclosure.

FIG. 2 shows a plot illustrating a relationship between temperature and the alveolar concentration of the indicated liquid anesthetics.

FIG. 3 shows a plot illustrating a sustained delivery of sevoflurane at minimal alveolar concentration over a one-hour time period at 16° C.

FIG. 4 shows an example graph of output concentration versus flow rate at a set output concentration for an anesthetic vaporizer in accordance with the disclosure.

FIG. 5 shows a graph of mouse core temperature over 30 minutes of anesthesia delivered using a standard variable bypass vaporizer versus the anesthetic vaporizer disclosed herein.

FIG. 6 shows a graph of mouse heart rate over 30 minutes of anesthesia delivered using a standard variable bypass vaporizer versus the anesthetic vaporizer disclosed herein.

FIG. 7 shows an example method of anesthetizing a subject in accordance with the disclosure.

FIG. 8 schematically shows an example computing system in accordance with the disclosure.

### DETAILED DESCRIPTION

The following detailed description is directed to systems and methods for delivery of anesthetic vapor that use the temperature-vapor pressure relationships of volatile anesthetics to deliver clinically relevant concentrations of anesthetic vapor. In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents. Various operations may be described as multiple discrete operations in turn, in a manner that may be helpful in understanding embodiments; however, the order of description should not be construed to imply that these operations are order dependent.

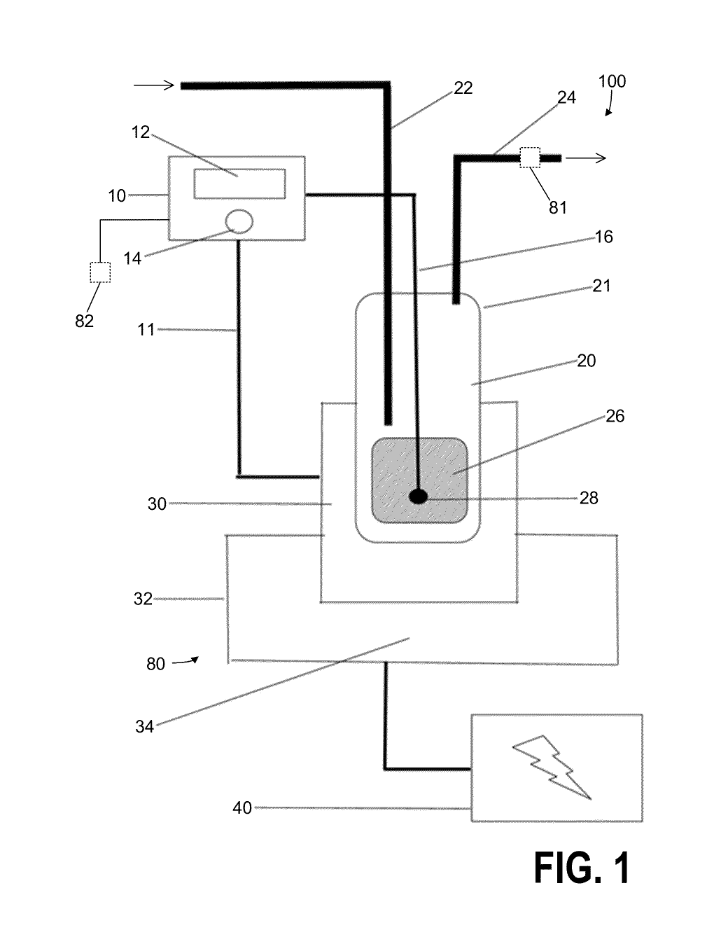

In one example embodiment—illustrated schematically in FIG. 1—a vaporizer 100 comprises a vessel 20 for the liquid anesthetic 26. The vessel further comprises a fresh gas inlet 22, a mixed gas outlet, 24 and a temperature sensor or probe 28. The temperature probe 28 may be coupled to or in communication with a controller 10 in any suitable way. For example, the temperature probe 28 may be in communication with controller 10 via wire 16. In additional examples, the vessel may further include a pressure sensor 82 that measures atmospheric pressure. Such an atmospheric pressure sensor 82 may be coupled to or in communication with controller 10.

The vaporizer 100 may additionally include a refrigeration system 80 comprising a cooling apparatus 30 that allows efficient transfer of heat from the vessel 20. The refrigeration system 80 is configured to cool the liquid anesthetic in the vessel to less than 0° C. In some examples, the refrigeration system may be configured to cool the vessel to less than 40° C. The refrigeration system may further comprise a cooling bath 32 that surrounds the cooling apparatus 30, and a power source 40. For example, the cooling apparatus 30 may be configured to receive the vessel 20 and the power source 40. In some examples, the cooling apparatus 30 may comprise a Peltier thermoelectric cooling device or any other suitable cooling device. In some examples, the power source 40 may be controlled using pulse-width modulation received from controller 10.

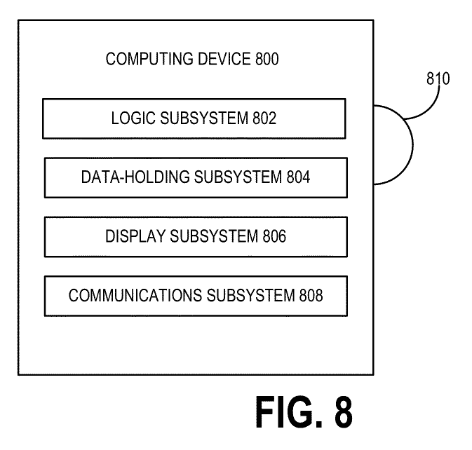

Controller 10 may comprise any suitable controlling device or computing system. For example, the controller 10 may comprise an electronic controlling device and/or may comprise a computing device or system as described below with regard to FIG. 8. The controller 10 may be configured to perform various operations, examples of which are described herein. The controlling device can further comprise a microprocessor that can receive temperature input, control the refrigeration system to achieve a target temperature, and manage a user interface. For example, the controller may be configured to receive input, output data, e.g., output data to a display device, and may include one or more processors having physical circuitry programmed to perform various operations described herein. The controller 10 may be configured to provide a user interface or may be coupled to various input devices that allow a user to set a desired concentration of the anesthetic in the mixed gas outlet. Additionally, the controller 10 may be configured perform an algorithm that calculates the appropriate set temperature to achieve the set concentration. Further, the controller may be configured to implement a proportional-integral-derivative (PID) controller to maintain the set temperature that in turn maintains the set concentration. The controller 10 may additionally include or be coupled to a display device 12, e.g., a screen, monitor, LED display, etc., to communicate information to the user. The controller 10 may additionally include or be coupled to one or more input devices, e.g., input device 14, for the user to input desired parameters to the controller. If the vessel comprises a sensor that measures atmospheric pressure, e.g., pressure sensor 82, then the controller may additionally be configured to implement an algorithm that changes the temperature of the vessel in response to raised or lowered atmospheric pressure in order to maintain the liquid anesthetic in the vessel at the temperature of minimal alveolar concentration. For example, in response to an increase in atmospheric pressure as measured by sensor 82, the controller may be configured to increase the temperature of the vessel in order to maintain the liquid anesthetic in the vessel at the temperature of minimal alveolar concentration. Conversely, in response to a decrease in atmospheric pressure as measured by sensor 82, the controller may be configured to decrease the temperature of the vessel in order to maintain the liquid anesthetic in the vessel at the temperature of minimal alveolar concentration

The vessel 20 may be made of any material inert enough not to react with chlorofluorocarbon anesthetics and which has a high thermal conductivity to promote transfer of heat from the anesthetic to the refrigeration system. In some examples, the vessel may be composed of a suitable metal such as aluminum. In further examples, the vessel may be insulated. The vessel may be insulated in any suitable way. For example, the vessel may be insulated by any method of insulation known in the art including the addition of an insulating material to the outside of the vessel by encasing the vessel in the insulating material permanently or removably, the construction of a double walled vessel filled with air, water, or another insulating material, or the construction of a vessel with more material than would be necessary to contain a chlorofluorocarbon anesthetic. In some examples, any part of the vessel not in contact with the refrigeration system may be insulated in such a way as to substantially prevent thermal transfer to the air, or any other component of the device but the refrigeration system.

In some examples, the vessel 20 may further include a lid 21. The lid may be removable or fused to the top or side of the vessel. The lid may further comprise openings or apertures configured to accept the fresh gas inlet 22 and the mixed gas outlet 24. The fresh gas inlet 22 may terminate inside the vessel at any point, including at any point above or below the level of the liquid anesthetic 26. In some examples, the fresh gas inlet may terminate inside the vessel below a minimal level of the liquid anesthetic such that fresh gas from the fresh gas inlet would bubble through the liquid anesthetic. The mixed gas outlet 24 may terminate at any point inside the vessel. In some examples, the mixed gas outlet 24 may terminate inside the vessel adjacent to the opening in the vessel through which it passes. For example, the mixed gas outlet may terminate inside the vessel at or about immediately after the opening. The inlet 22 and outlet 24 may have any suitable shape that efficiently allows flow of gas. For example, the inlet 22 and the outlet may be shaped as a tube or any other structure comprising a lumen. The inlet 22 and outlet 24 may be composed of any suitable material such as any kind of plastic or metal, or other material known in the art. In some examples, the inlet 22 and outlet 24 may be coupled to the inside and/or the outside of the lid so as to provide an airtight seal around the openings. For example, gaskets or other sealing members may be included at the interfaces of the inlet and outlet with the openings in the vessel or lid.

The inlet and/or outlet and/or vessel may further include valves and other control mechanisms used in regulating the flow of the anesthetic including shut off valves, pressure release valves, wicking systems, other adjustment systems, and other features, many of which are well known in the art. For example, such features are described in patent applications including: U.S. Pat. No. 2,869,540, U.S. Pat. No. 2,941,528, U.S. Pat. No. 3,106,917, U.S. Pat. No. 3,534,732, U.S. Pat. No. 3,528,418, U.S. Pat. No. 3,651,805, U.S. Pat. No. 3,841,560, U.S. Pat. No. 4,067,935, U.S. Pat. No. 4,075,297, U.S. Pat. No. 4,059,657, U.S. Pat. No. 4,129,621, U.S. Pat. No. 4,693,853, U.S. Pat. No. 4,879,997, U.S. Pat. No. 5,535,737, U.S. Pat. No. 5,664,561, U.S. Pat. No. 7,992,843, U.S. Pat. No. 8,448,638, US Application Number 20100180893, all of which are incorporated by reference herein.

Additionally, in some examples, the mixed gas outlet 24 may pass over or through a heating element 81 which is configured to increase the temperature of cooled anesthetic vapor output from vaporizer 100. In particular, the heating element 81 may be configured to increase the temperature of cooled anesthetic vapor output via mixed gas outlet 24 to a predetermined threshold temperature before the anesthetic vapor is delivered to a subject. The heating element 81 may comprise any suitable element or device which generates and transfers heat to the cooled anesthetic vapor output from vaporizer 100. As an example, heating element 81 may comprise a warming mat set to a predetermined temperature, e.g., 37° C. As another example, heating element 81 may comprise tubing wrapped around the mixed gas outflow, where said tubing contains fluid heated to a controlled temperature. As yet another example, heating element 81 may comprise a resistive heating element wrapped around or in contact with the mixed gas flow and heated to a controlled temperature. In some examples, the heating element 81 may be coupled to controller 10 and user input received by controller 10 may be used to set a temperature threshold of the heating element. In some examples, heating element 81 may be omitted and the temperature of the cooled anesthetic vapor output via the mixed gas outlet may be increased by exposure to room temperature prior to delivery to the subject.

In some examples, the temperature sensor 28 may be integral to the vessel 20 and thereby connected to a wire 16 or other suitable connection via a plug external to the vessel. Alternatively, the sensor may float inside the vessel and be connected directly to the controlling device 10 by the wire 16 through an opening in the vessel. The temperature sensor may be configured to communicate temperature information to the controlling device which in turn controls the refrigeration system.

The refrigeration system can be any refrigeration system that can cool a liquid anesthetic to a temperature below 0° C. For example, the refrigeration system may be configured to cool a liquid anesthetic to a temperature below −60° C. In some examples, the refrigeration system also can rapidly warm the anesthetic to adjust the concentration of the anesthetic upwards. In some examples, the refrigeration system may comprise a thermoelectric cooling system such as a Peltier cooling system. In other examples, the cooling system may comprise a compression/expansion refrigeration system which includes a heating element.

### EXAMPLES

The following examples are illustrative of disclosed methods. In light of this disclosure, those of skill in the art will recognize that variations of these examples and other examples of the disclosed method would be possible without undue experimentation. It should be understood that the examples described below are provided for illustrative purposes and are not intended to be limiting.

### Example 1

### Introduction

The temperature-vapor pressure relationship for desflurane, isoflurane, and sevoflurane is well described at temperatures greater than 0 degrees Celsius (° C.) (Susay S R et al, Anesth Analg 83, 864-866 (1996); incorporated by reference herein) but has not been previously described at temperatures less than 0° C. Because the production of vapor leads to decrease in temperature, which in turn leads to a decrease in saturated vapor pressure, existing variable-bypass vaporizers are temperature-compensated and contain significant thermal mass to prevent inconsistency in the rate of vapor production caused by changes in fresh gas, ambient, and vaporizer temperatures. The saturated vapor pressures of anesthetic agents above 0° C. are much higher than those used in the clinical setting.

Described herein is the achievement of clinically relevant concentrations of anesthetic vapors at temperatures of less than 0° C. This was achieved by determining the temperature of an anesthetic agent at 1 atmosphere of pressure at which the minimum alveolar concentration (MAC) of the agent is delivered. This temperature can be referred to as the “T_{MAC}” of the agent. Once the temperature-vapor pressure relationship at clinical concentrations is determined, the information can be used to construct a functional anesthetic vaporizer with digital temperature control, as opposed to variable bypass at constant temperature.

### Example 2

### Methods

To determine the temperature at which the minimal alveolar concentration of a volatile anesthetic composition is delivered (T_{MAC}), a volatile anesthetic agent can be placed in a glass Erlenmeyer flask. The Erlenmeyer flask is partially submerged in a dry ice bath. A fresh-air inflow line (with a flow meter) can be used to provide a constant flow of fresh air at 1 liter/minute. Waste vapor can be exhausted into a fume hood. A gas sample line can be connected to an anesthesia gas monitor (POET II®, Criticare Systems, Inc., Waukesha, Wis.) to determine anesthetic concentrations. Agent temperature can be measured, and anesthetic concentrations can be recorded at intervals of 1° C. as the agent is slowly cooled.

The described method was performed using desflurane, isoflurane, and sevoflurane and the method was repeated 5 times for each agent. Plots of anesthetic concentration versus temperature were created using statistical software (GraphPad Software, La Jolla, Calif.). Data was predicted by a cubic curve.

After determining T_{MAC} for each of the three agents, an anesthetic vaporizer was constructed that delivered a user-set anesthetic concentration determined by the temperature of the agent. Unlike a variable-bypass design, the full volume of fresh gas flowed through the vaporizer chamber. Using a temperature sensor, digital microcontroller, and custom software, the vaporizer actively controlled the temperature of the volatile agent reservoir using pulse-width-modulation of current delivered to a Peltier thermoelectric cooling device.

### Example 3

### T_{MAC} Determination

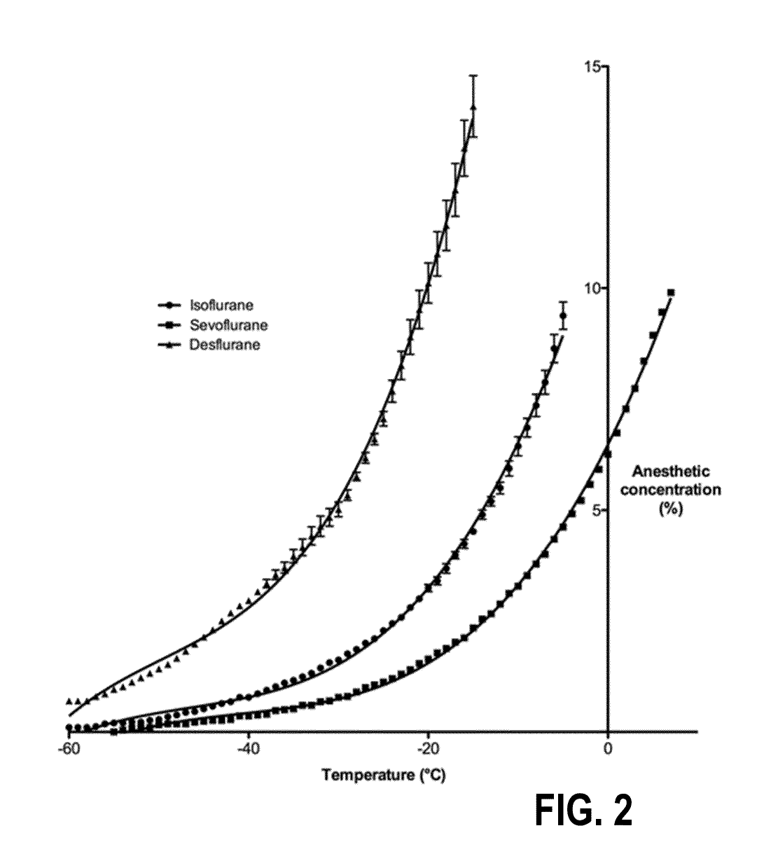

Each of the agents was successfully cooled to a temperature of less than −50° C. (FIG. 2) The cooling-concentration relationship was consistent in multiple trials. Curves were fit and the temperature-saturated vapor pressure relationship was y=0.0002x³+0.0301x²+1.6018x+31.605 for desflurane (r²=0.9985), y=0.0001x³+0.0137x²+0.6706x+11.9598 for isoflurane (r²=0.9977), and y=0.00008x³+0.0093x²+0.4024x+6.48 for sevoflurane (r²=0.9999). The T_{MAC} of desflurane was calculated to be −26° C. (6.6±0.2%, n=5), isoflurane, −35° C. (Anesthetic concentration 1.1±0.04%, n=5), and sevoflurane, −16±° C. (2.2±0.02%, n=5).

### Example 4

### Vaporizer Validation

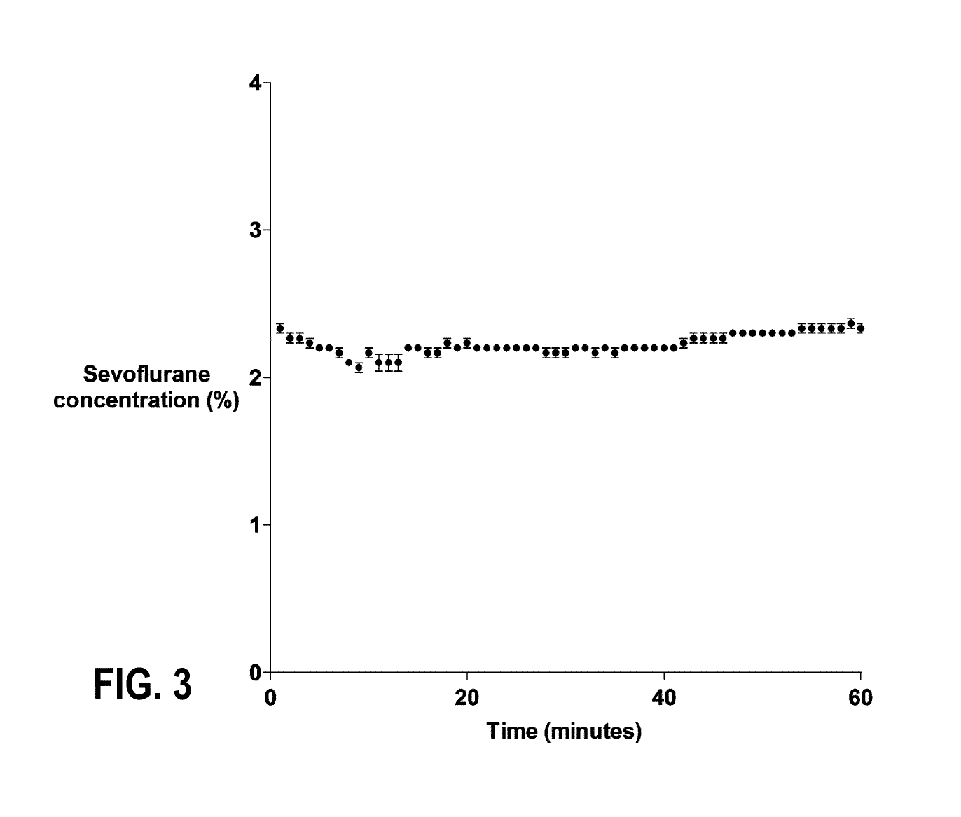

After the temperature-controlled vaporizer was constructed, the vessel was filled with sevoflurane and the desired anesthetic temperature was set to −16° C., the experimentally derived T_{MAC} of sevoflurane. Anesthetic concentrations and temperatures were recorded over a one-hour period to determine whether the vaporizer was able to maintain the delivery of the desired anesthetic concentration. The vaporizer sustained delivery of 1 MAC (2.2±0.02%, n=3) of sevoflurane for 1 hour at the T_{MAC} of −16° C. (FIG. 3)

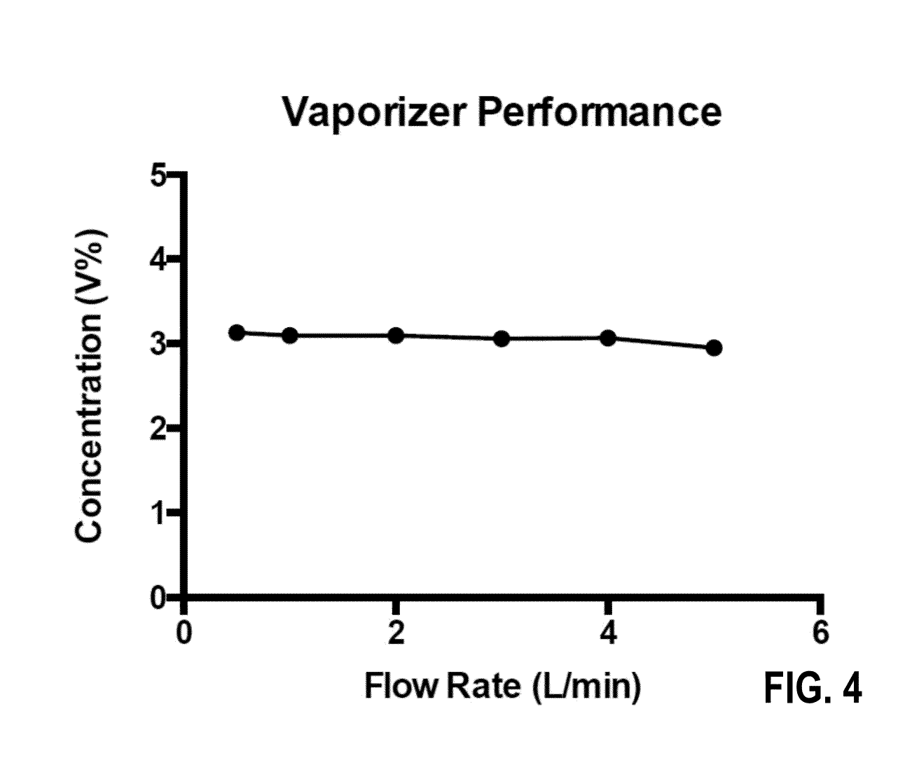

As another example, FIG. 4 shows an example graph of an anesthetic vaporizer output concentration (sevoflurane) versus flow rate at a set output concentration (3.0 V/V%) for an anesthetic vaporizer in accordance with the disclosure. In particular, FIG. 4 shows a graph of vaporizer output, e.g., for the vaporizer 100 described above, for a set concentration of 3.0 MAC at multiple flows. The results shown in FIG. 4 demonstrate a sustained and consistent delivery of the desired anesthetic concentration.

### Example 5

### Vaporizer Validation

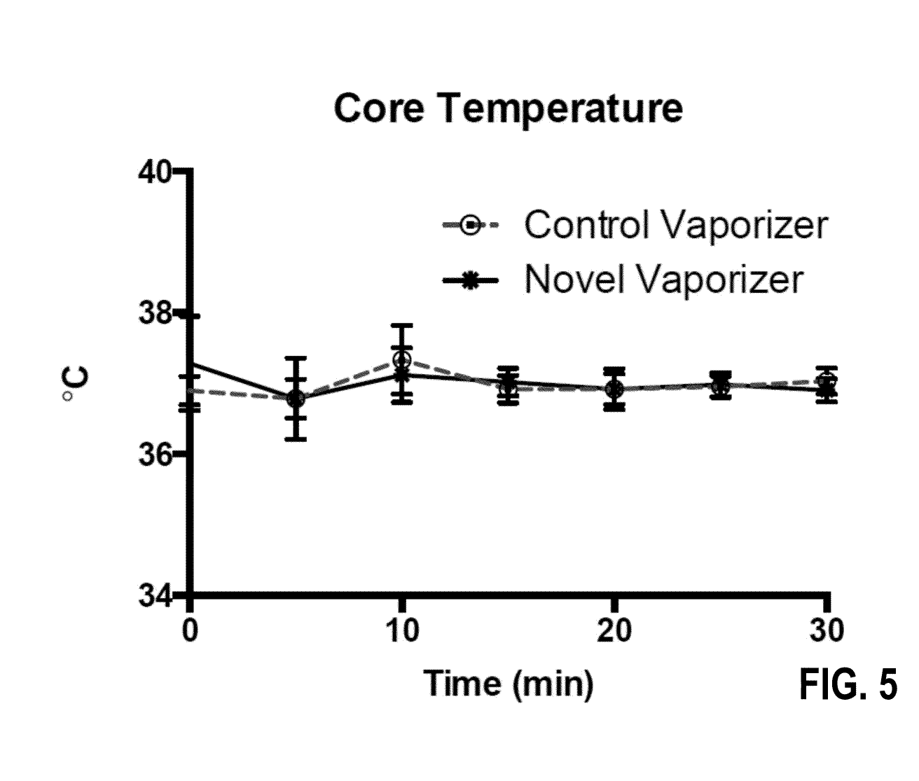

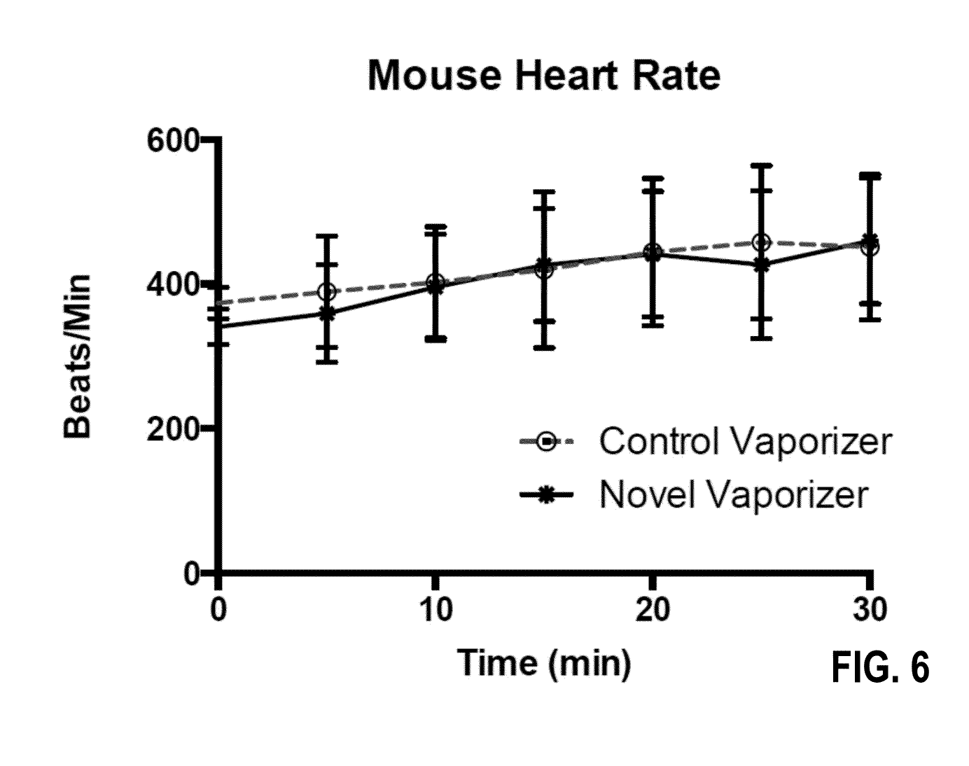

As remarked above, in some examples, the cooled anesthetic vapor output by vaporizer 100 may be warmed up or heated, e.g., via heating element 81 described above, prior to administering the anesthetic vapor to a subject. Warming or heating the cooled anesthetic vapor output by vaporizer 100 may not substantially change the concentration of the anesthetic vapor. In particular, the concentration of the anesthetic vapor output from the vessel 20 of vaporizer 100 via mixed gas outlet 24 may stay substantially the same after passing over or through heating element 81 before delivery to a subject. By warming or heating the cooled anesthetic vapor output by vaporizer 100 before delivery to a subject, the temperature of the anesthetic vapor may be increased to a temperature suitable for delivery to the subject. For example, FIG. 5 shows a graph of mouse core temperature over 30 minutes of anesthesia delivered using a standard variable bypass vaporizer versus the anesthetic vaporizer disclosed herein. In particular, FIG. 5 shows mouse core temperature over 30 minutes of anesthesia delivered using either vaporizer 100 or a standard variable bypass vaporizer, both set to the same concentration (3%). In this example, the outflow tubing of vaporizer 100 was laid across a warming mat set at 37 degrees Celsius, and the mice were supine on a surgical surface, with a standard, temperature controlled heating lamp. During this experiment, mouse heart rate (see FIG. 6), respiratory rate, and the temperature of the gas they were breathing were also measured. As demonstrated by this data, there was no detectable difference in physiology between mice anesthetized with vaporizer 100, and those anesthetized with an identical sevoflurane concentration using traditional technology.

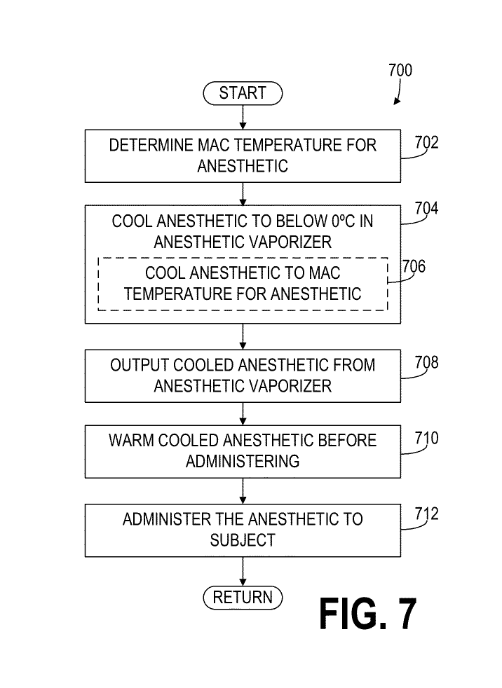

FIG. 7 shows an example method 700 of anesthetizing a subject in accordance with the disclosure. One or more steps of method 700 may be performed by anesthetic vaporizer 100 described above. Additionally, in some examples, one or more steps of method 700 may be implemented by a controller, e.g., controller 10 described above. Any suitable liquid anesthetic may be included in vaporizer 100. As one example, the liquid anesthetic included in the vaporizer may be selected from desflurane, isoflurane, and sevoflurane.

At 702, method 700 includes determining a MAC temperature, T_{MAC}, for the anesthetic. In some examples, a T_{MAC} value may be input or selected by a user via an input device or interface coupled to or included in controller 10. T_{MAC} may be determined as described above with regard to Example 3, for example.

At 704, method 700 includes cooling the anesthetic to below 0° C. in the anesthetic vaporizer 100. For example, anesthetic contained in vessel 20 may be cooled to below 0° C. via cooling apparatus 30. In some examples, at 706, method 700 may include cooling the anesthetic to the MAC temperature, T_{MAC}, for anesthetic. For example, if the liquid anesthetic in the vaporizer is desflurane, then the anesthetic may be cooled to approximately −26° C., which is the T_{MAC} for desflurane. As another example, if the liquid anesthetic in the vaporizer is isoflurane, then the anesthetic may be cooled to approximately −35° C., which is the T_{MAC} for isoflurane. As yet another example, if the liquid anesthetic in the vaporizer is sevoflurane, then the anesthetic may be cooled to approximately −16° C., which is the T_{MAC} for sevoflurane.

In some examples, during the cooling of the anesthetic, a temperature sensor, e.g., sensor 28 may be used to monitor the temperature of the anesthetic in vaporizer 100. Sensor 28 may be in communication with controller 10 and controller 10 may be configured to provide an indication, e.g., via a display device, that the temperature of the anesthetic has been cooled to T_{MAC}.

At 708, method 700 includes outputting the cooled anesthetic from the anesthetic vaporizer. For example, the cooled anesthetic in vessel 20 may be output from vessel 20 via mixed gas outlet 24. At 710, method 700 may optionally include heating cooled anesthetic output by the anesthetic vaporizer prior to administering the cooled anesthetic to the subject. For example, the cooled anesthetic may pass over or through heating element 81. Heating element 81 may be configured to increase the temperature of anesthetic vapor output from the vessel 20 via the mixed gas outlet 24. In particular, heating element 81 may be configured to provide a predetermined amount of heat to the cooled anesthetic vapor such that the temperature of the cooled anesthetic vapor is increased to a predetermined temperature suitable for delivery to a subject. At 712, method 700 includes administering the anesthetic to the subject.

In some embodiments, the above described methods and processes may be tied to a computing system, including one or more computers. In particular, the methods and processes described herein, e.g., method 700 described above, may be implemented as a computer application, computer service, computer API, computer library, and/or other computer program product.

FIG. 8 schematically shows a nonlimiting computing device 800 that may perform one or more of the above described methods and processes. For example, computing device 800 may represent controller 10 shown in FIG. 1 described above. As such computing device 800 may be configured to perform various operations such as receiving user input to set a first concentration of an anesthetic, calculating a set temperature to achieve the temperature of minimal alveolar concentration of the anesthetic, implementing a proportional-integral-derivative controller to maintain the liquid anesthetic at the set temperature, and adjusting the set temperature to achieve the temperature of minimal alveolar concentration based upon an atmospheric pressure reading received from a pressure sensor.

Computing device 800 is shown in simplified form. It is to be understood that virtually any computer architecture may be used without departing from the scope of this disclosure. In different embodiments, computing device 800 may take the form of a microcomputer, an integrated computer circuit, microchip, a mainframe computer, server computer, desktop computer, laptop computer, tablet computer, home entertainment computer, network computing device, mobile computing device, mobile communication device, gaming device, etc.

Computing device 800 includes a logic subsystem 802 and a data-holding subsystem 804. Computing device 800 may optionally include a display subsystem 806 and a communication subsystem 808, and/or other components not shown in FIG. 8. Computing device 800 may also optionally include user input devices such as manually actuated buttons, switches, keyboards, mice, game controllers, cameras, microphones, and/or touch screens, for example.

Logic subsystem 802 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. Additionally or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 804 may include one or more physical, non-transitory, devices configured to hold data and/or instructions executable by the logic subsystem to implement the herein described methods and processes. When such methods and processes are implemented, the state of data-holding subsystem 804 may be transformed (e.g., to hold different data).

Data-holding subsystem 804 may include removable media and/or built-in devices. Data-holding subsystem 804 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 804 may include devices with one or more of the following characteristics: volatile, nonvolatile, dynamic, static, read/write, read-only, random access, sequential access, location addressable, file addressable, and content addressable. In some embodiments, logic subsystem 802 and data-holding subsystem 804 may be integrated into one or more common devices, such as an application specific integrated circuit or a system on a chip.

FIG. 8 also shows an aspect of the data-holding subsystem in the form of removable computer-readable storage media 810, which may be used to store and/or transfer data and/or instructions executable to implement the herein described methods and processes. Removable computer-readable storage media 810 may take the form of CDs, DVDs, HD-DVDs, Blu-Ray Discs, EEPROMs, flash memory cards, and/or floppy disks, among others.

When included, display subsystem 806 may be used to present a visual representation of data held by data-holding subsystem 804. As the herein described methods and processes change the data held by the data-holding subsystem, and thus transform the state of the data-holding subsystem, the state of display subsystem 806 may likewise be transformed to visually represent changes in the underlying data. Display subsystem 806 may include one or more display devices utilizing virtually any type of technology. Such display devices may be combined with logic subsystem 802 and/or data-holding subsystem 804 in a shared enclosure, or such display devices may be peripheral display devices.

When included, communication subsystem 808 may be configured to communicatively couple computing device 800 with one or more other computing devices. Communication subsystem 808 may include wired and/or wireless communication devices compatible with one or more different communication protocols. As nonlimiting examples, the communication subsystem may be configured for communication via a wireless telephone network, a wireless local area network, a wired local area network, a wireless wide area network, a wired wide area network, etc. In some embodiments, the communication subsystem may allow computing device 800 to send and/or receive messages to and/or from other devices via a network such as the Internet.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. An anesthetic vaporizer, comprising:
a vessel configured to contain a liquid anesthetic, the vessel further comprising a fresh gas inlet, a mixed gas outlet, and a temperature probe;
a refrigeration system configured to cool the liquid anesthetic in the vessel to less than 0° C., the refrigeration system further comprising a cooling apparatus configured to receive the vessel and a power source; and
an electronic controlling device.

2. The vaporizer of claim 1, wherein the electronic controlling device is configured to perform one or more of the following operations: provide an interface configured to allow a user to set a first concentration of the anesthetic; implement an algorithm that calculates a set temperature to achieve the temperature of minimal alveolar concentration of the anesthetic; and implement a proportional-integral-derivative controller to maintain the liquid anesthetic at the set temperature.

3. The vaporizer of claim 2, further comprising an atmospheric pressure sensor, and wherein the electronic controlling device is further configured to adjust the set temperature to achieve the temperature of minimal alveolar concentration based upon the atmospheric pressure.

4. The vaporizer of claim 1, wherein the refrigeration system is configured to cool the vessel to less than 40° C.

5. The vaporizer of claim 1, wherein the power source is controlled by pulse-width modulation.

6. The vaporizer of claim 1, wherein the cooling apparatus is a Peltier thermoelectric cooling device.

7. The vaporizer of claim 1, further comprising a cooling bath.

8. The vaporizer of claim 1, wherein the vessel is insulated.

9. The vaporizer of claim 1, wherein the liquid anesthetic is selected from desflurane, isoflurane, and sevoflurane.

10. The vaporizer of claim 1, wherein the fresh gas inlet is positioned to allow fresh gas to bubble through the liquid anesthetic.

11. A method of anesthetizing a subject, the method comprising:
cooling the anesthetic to below 0° C.; and
administering the cooled anesthetic to the subject using an anesthetic vaporizer.

12. The method of claim 11, further comprising cooling the anesthetic to the temperature of minimal alveolar concentration for the anesthetic.

13. The method of claim 12, wherein the anesthetic is desflurane and the temperature of minimal alveolar concentration is approximately −26° C.

14. The method of claim 12, wherein the anesthetic is isoflurane and the temperature of minimal alveolar concentration is approximately −35° C.

15. The method of claim 12, wherein the anesthetic is sevoflurane and wherein the temperature of minimal alveolar concentration is approximately −16° C.

16. The method of claim 11, further comprising determining the temperature of minimal alveolar concentration for the anesthetic.

17. The method of claim 11, further comprising heating cooled anesthetic output by the anesthetic vaporizer prior to administering the cooled anesthetic to the subject.

18. An anesthetic vaporizer, comprising:
a vessel configured to contain a liquid anesthetic, the vessel further comprising a fresh gas inlet, a mixed gas outlet, and a temperature probe;
a refrigeration system configured to cool the liquid anesthetic in the vessel to less than 0° C., the refrigeration system further comprising a cooling apparatus configured to receive the vessel and a power source; and
a controller configured to:
receive user input to set a first concentration of the anesthetic;
calculate a set temperature to achieve the temperature of minimal alveolar concentration of the anesthetic; and
implement a proportional-integral-derivative controller to maintain the liquid anesthetic at the set temperature.

19. The vaporizer of claim 18, further comprising an atmospheric pressure sensor and wherein the controller is further configured to adjust the set temperature to achieve the temperature of minimal alveolar concentration based upon the atmospheric pressure.

20. The vaporizer of claim 18, further comprising a heating element configured to increase the temperature of anesthetic vapor output from the vessel.
